(19) United States
(12) Patent Application Publication
(10) Pub. No.: US 2024/0341687 A1
(43) Pub. Date: Oct. 17, 2024

(54) CATHETERS WITH REINFORCED SEGMENT AND ATTACHED TUBE

(71) Applicant: Boston Scientific Scimed, Inc., Maple Grove, MN (US)

(72) Inventors: Harshil Kacha, Burlington (CA); Miles Montgomery, Holland Landing (CA); Ros Brannick, Picton (CA); Suresh Subramaniam, Cambridge (CA)

(21) Appl. No.: 18/626,677

(22) Filed: Apr. 4, 2024

Related U.S. Application Data
(60) Provisional application No. 63/495,747, filed on Apr. 12, 2023.

(51) Int. Cl. A61B 5/00; A61B 5/06; A61B 5/283; A61B 5/367; A61B 18/00; A61B 18/14; A61M 25/00; A61M 25/01; A61M 29/00

(52) U.S. Cl. CPC A61B 5/6852; A61B 5/063; A61B 5/283; A61B 5/367; A61B 18/1492; A61B 2018/00577; A61M 25/0013; A61M 25/0051; A61M 2025/0166; A61M 29/00

(57) ABSTRACT

Catheters including an elongated shaft defining a lumen along a longitudinal axis and having a distal portion are disclosed. The elongated shaft includes a longitudinally extending inner member defining the lumen, a support member disposed on the longitudinally extending inner member, and a reinforcement member. The support member includes a proximal portion and a distal portion separated by a breakout section. The reinforcement member is disposed on the breakout section and having a reinforcement proximal end and a reinforcement distal end, the reinforcement proximal end coupled to the proximal portion and the reinforcement distal end coupled to the distal portion. An elongated tube extends along the shaft. The tube extends longitudinally along the proximal braided member radially underneath the proximal braided member, extends longitudinally on the outer surface along the distal braided member, and extends from underneath the proximal braided member to the outer surface at the breakout section.

### CROSS REFERENCE TO RELATED APPLICATIONS

The present application claims priority of U.S. Provisional Patent Application 63/495,747 entitled “CATHETERS WITH REINFORCED SEGMENT AND ATTACHED TUBE,” filed Apr. 12, 2023, which is hereby incorporated by reference in its entirety.

### TECHNICAL FIELD

The present disclosure relates to medical devices for catheterization procedures, such as medical devices for electrophysiological procedures. More specifically, the present disclosure relates to catheters, catheter systems, and methods for manufacturing catheters.

### BACKGROUND

Various medical fields use different types of catheters to achieve access to a physiological site in medical procedures. For instance, electrophysiological procedures involve guiding catheters into the heart and tracking the location of the catheters with respect to the heart. Catheter ablation is minimally invasive electrophysiological procedure to treat a variety of heart conditions such as supraventricular and ventricular arrhythmia. Example catheters used in catheter ablation can include mapping catheters, ablation catheters, guiding sheaths, dilators, and other medical tools, which can be referred to as catheters in this disclosure. Electrophysiological procedures can involve the visualization of the heart, heart activity, and the position of the catheters within the heart. A common visualization system involves the use of fluoroscopy, which can expose the patient and clinician to radiation. Electroanatomical mapping is an alternative visualization technique that does not involve the use of radiation.

Electroanatomical mapping allows a clinician to accurately determine the location of an arrhythmia, define cardiac geometry in three dimensions, delineate areas of anatomic interest, and permits imaging of the catheters for positioning and manipulation. Catheters used with electroanatomical mapping systems can include tracking capabilities, such as navigation enabled or impedance-based tracking methodologies. Navigation-enabled catheters use magnetic sensors in the presence of magnetic fields to track the location and orientation of the catheters. But not all catheters include magnetic sensors. Impedance-based catheters use electrodes in the presence of electric fields to track the catheters.

### SUMMARY

In an Example 1, a catheter comprising an elongated shaft defining a lumen along a longitudinal axis and having a distal section; the shaft comprising: a longitudinally extending inner member defining the lumen; a support member disposed on the longitudinally extending inner member, the support member including a proximal portion and a distal portion separated by a breakout section, the distal portion having an outer surface; and a reinforcement member disposed at the breakout section and having a reinforcement proximal end and a reinforcement distal end, the reinforcement proximal end coupled to the proximal portion and the reinforcement distal end coupled to the distal portion; an electrode disposed on the distal portion of the elongated shaft; an elongated tube extending longitudinally along the elongated shaft; and a lead conductor disposed within the tube and electrically coupled to the electrode; wherein the tube extends longitudinally along the proximal portion radially underneath the proximal portion, extends longitudinally on the outer surface along the distal portion, and extends from underneath the proximal portion to the outer surface at the reinforcement member.

In an Example 2, the catheter of Example 1, wherein the support member includes a stainless-steel braided material.

In an Example 3, the catheter of Example 2, wherein the braided material includes conductive fibers.

In an Example 4, the catheter of any of Examples 1-3, wherein the elongated shaft further comprises a reinforcement member having a tubular longitudinal side, a reinforcement proximal end, and a reinforcement distal end, the reinforcement proximal end operably coupled to the proximal portion and the reinforcement distal end operably coupled to the distal portion.

In an Example 5, the catheter of Example 4, wherein the reinforcement member includes one of a framed member having a plurality of first annular members attached to and spaced apart by a plurality of longitudinally extending cross members and a tubular coiled member having a plurality of second annular members attached to and spaced apart by a longitudinally extending coil.

In an Example 6, the catheter of any of Examples 4-5, wherein the longitudinal side extends along the axis and an opening is formed on the longitudinal side, and wherein the tube extends from underneath the proximal portion to the outer surface through the opening of the reinforcement member.

In an Example 7, the catheter of any of Examples 4-6, wherein the reinforcement member is formed from a laser machined tube.

In an Example 8, the catheter of any of Examples 1-3, wherein the reinforcement member includes a thread wound around the tube and breakout section.

In an Example 9, the catheter of any of claims 1-8, wherein the shaft includes an outer layer disposed on the support member and the reinforcement member.

In an Example 10, the catheter of claim 9, wherein the outer layer includes a polyether block amide.

In an Example 11, the catheter of Example 1, wherein the electrode is ring electrode.

In an Example 12, the catheter of any of Examples 1-11, wherein the electrode includes a plurality of electrodes, and the tube includes a plurality of tubes.

In an Example 13, the catheter of any of Examples 1-12, wherein the shaft includes a proximal end, the proximal end operably coupled to an electrical connector, and the lead conductor electrically coupled to the electrical conductor.

In an Example 14, the catheter of any of Examples 1-13, wherein the proximal braided member includes an inner surface, and the tube extends longitudinally along the inner surface.

In an Example 15, the catheter of any of Examples 1-14, wherein the catheter is a guide catheter having a proximal end coupled to a handle.

In an Example 16, a catheter comprising an elongated shaft defining a lumen along a longitudinal axis and having a distal section; the shaft comprising: a longitudinally extending inner member defining the lumen; a support member disposed on the longitudinally extending inner member, the support member including a proximal portion and a distal portion separated by a breakout section, the distal portion having an outer surface; and a reinforcement member disposed at the breakout section and having a reinforcement proximal end and a reinforcement distal end, the reinforcement proximal end coupled to the proximal portion and the reinforcement distal end coupled to the distal portion; an electrode disposed on the distal portion of the elongated shaft; an elongated tube extending longitudinally along the elongated shaft; and a lead conductor disposed within the tube and electrically coupled to the electrode; wherein the tube extends longitudinally along the proximal portion radially underneath the proximal portion, extends longitudinally on the outer surface along the distal portion, and extends from underneath the proximal portion to the outer surface at the reinforcement member.

In an Example 17, the catheter of cl Example aim 16, wherein the support member is formed of a braided material.

In an Example 18, the catheter of Example 16, wherein the proximal braided member and the distal braided member include conductive fibers.

In an Example 19, the catheter of Example 16, wherein the elongated shaft further comprises a reinforcement member having a tubular longitudinal side, a reinforcement proximal end, and a reinforcement distal end, the reinforcement proximal end operably coupled to the proximal portion and the reinforcement distal end operably coupled to the distal portion.

In an Example 20, the catheter of Example 19, wherein the reinforcement member includes one of a framed member having a plurality of first annular members attached to and spaced apart by a plurality of longitudinally extending cross members and a tubular coiled member having a plurality of second annular members attached to and spaced apart by a longitudinally extending coil.

In an Example 21, the catheter of Example 20, wherein the reinforcement member is formed from a laser machined tube.

In an Example 22, the catheter of Example 19, wherein the longitudinal side extends along the axis and an opening is formed on the longitudinal side, and wherein the tube extends from underneath the proximal portion to the outer surface through the opening of the reinforcement member.

In an Example 23, the catheter of Example 16, wherein the reinforcement member includes a thread wound around the tube and breakout section.

In an Example 24, the catheter of Example 16, wherein the shaft includes an outer layer disposed on the support member and the reinforcement member.

In an Example 25, the catheter of Example 16, wherein the elongate tube includes a plurality of elongate tubes radially spaced-apart on the outer side of the distal portion.

In an Example 26, the catheter of Example 16, wherein the inner member includes in inner surface, and the inner surface includes a liner layer.

In an Example 27, the catheter of Example 16, wherein the catheter includes one of a dilator and a guide catheter.

In an Example 28, the catheter of Example 16, wherein the shaft includes a distal tip section having an ablation electrode assembly.

In an Example 29, the catheter of Example 16, wherein the electrode is a ring electrode.

In an Example 30, a method of manufacturing a catheter, the method comprising: forming a distal portion of an elongated member from an inner member defining a lumen, a proximal braided member, and a distal braided member spaced apart from the proximal braided member at a breakout section, the proximal braided member and distal braided member disposed on the inner member; attaching an elongated tube extending longitudinally along the elongated member and within an inner side of the proximal braided member, extending longitudinally along an outer side of the distal braided member, and through breakout section; forming a reinforcement member on the breakout section; and disposing an outer layer on the elongated member to form a shaft.

In an Example 31, the method of Example 30, wherein the reinforcement member is formed by winding a thread over the elongated member.

In an Example 32, a system for tracking a catheter during an electrophysiology procedure on a patient, the system comprising: a patch electrode mechanically couplable to the patient; a catheter disposable within a patient, the catheter comprising: an elongated shaft defining a lumen along a longitudinal axis and having a distal portion; the shaft comprising: a proximal braided member comprising a first braided member distal end; a distal braided member having an outer surface and a second braided member proximal end, the second braided member proximal end longitudinally spaced-apart from the first braided member distal end; and a reinforcement member having an aperture, a reinforcement proximal end, and a reinforcement distal end, the reinforcement proximal end operably coupled to the first braided member distal end and the reinforcement distal end operably coupled to the second braided member proximal end; an electrode disposed on the distal portion of the elongated shaft; an elongated tube extending longitudinally along the elongated shaft; and a lead conductor disposed within the tube and electrically coupled to the electrode; wherein the tube extends longitudinally along the proximal braided member radially underneath the proximal braided member, extends longitudinally on the outer surface along the distal braided member, and extends from within the lumen to the outer surface through the aperture of the reinforcement member; and a controller operably coupled to the patch electrode and the tracking electrode, the controller configured to receive the electrical signal and determine a position of the catheter with respect to the patient.

In an Example 33, the system of Example 32, wherein the controller if further configured to generate an electroanatomical map of the heart of the patient.

In an Example 34, the system of Example 32, wherein the controller is configured to determine the position of the catheter via an impedance-based process.

In an Example 35, the system of Example 32, wherein the reinforcement member is of a material dissimilar from the proximal braided member and the distal braided member.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, the example clinical setting having an example electrophysiology system.

FIG. 2 is a schematic diagram illustrating an example catheter that can be used with the example electrophysiology system. of FIG. 1.

FIG. 3A is a schematic diagram illustrating from a side view an example feature of the example catheter of FIG. 2.

FIG. 3B is a schematic diagram illustrating from a sectioned view the example feature of the example catheter of FIG. 3A.

FIG. 4A is a schematic diagram illustrating from a side view an example reinforcement member of the example catheter of FIG. 2.

FIG. 4B is a schematic diagram illustrating from a side view another example reinforcement member of the example catheter of FIG. 2.

FIG. 5 is a schematic diagram illustrating from a side view another example feature of the example catheter of FIG. 2.

FIG. 6 is a block diagram illustrating an example method of manufacturing the medical imaging device of FIG. 2.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

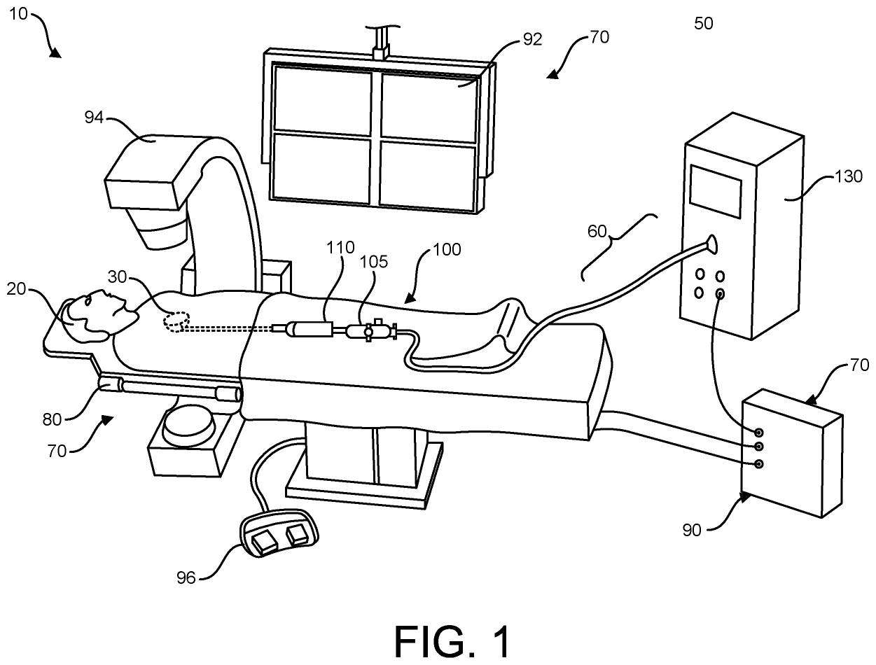

FIG. 1 illustrates an example clinical setting 10 for treating a patient 20, such as for treating a heart 30 of the patient 20, using an electrophysiology system 50, in accordance with the disclosure. The electrophysiology system 50 includes a catheter system 60 and an electroanatomical mapping (EAM) system 70. The example catheter system 60 includes an elongated catheter assembly 100, which includes an ablation catheter 105 and a catheter sheath 110, and an electroporation console 130. Additionally, the catheter system 60 includes various connecting elements, such as cables, that operably connect the components of the catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 can be navigated to within the patient's heart, such as within a chamber of the heart.

The example catheter system 60 is configured to deliver ablation energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheters. For instance, a catheter defines a longitudinal axis that passes through a centroid of a cross section of the catheter, such as the centroid of a cross section of a shaft of catheter 105 or a centroid of a cross section of a main lumen of a sheath 110. As shown, the catheter 105 is disposed within the sheath 110. The catheters 105, 110 are movable with respect to each other along the longitudinal axis.

The example catheter 105 includes an elongated catheter shaft and distal end configured to be deployed proximate to the target tissue, such as within a chamber of the patient's heart. The distal end may include a basket, balloon, spline, configured tip, or other deployment mechanism to effect treatment. The deployment mechanism can include an electrode assembly or array having a plurality of ablation electrodes. Each of the plurality of ablation electrodes is electrically coupled to a corresponding elongated lead conductor that extends along the shaft to a catheter proximal end. The lead conductors can be electrically coupled to a plug in the proximal region of the catheter 105, such as a plug configured to be mechanically and electrically coupled to the console 130, for example, either directly or via intermediary electrical conductors such as cabling. In one example, the console 130 is configured to provide an electrical signal, such as a plurality of concurrent or space-apart-time electrical signals, to the electrically connected catheter 105 along lead conductors to the spaced-apart electrodes to effect ablation.

The console 130 is configured to control aspects of the catheter system 60. The console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 can be operable to track the location of the various components of the catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 can generate a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter, or both. In these examples, the location sensing elements can constitute tracking electrodes on the tracked catheters that generate outputs received and processed by the mapping and navigation controller 90 to track the location of the various location sensing electrodes within the localization volume. For instance, an impedance tracking methodology can employ the use of a patch electrode (not shown) attached to the patient's body, a current or impedance based can be determined between the tracking electrode on the catheter and the patch electrode.

The EAM system 70 can be equipped for magnetic tracking capabilities, impedance tracking capabilities, or for both magnetic and impedance tracking capabilities. Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the case of impedance-based tracking with the EAM system 70, the catheters include tracking electrodes disposed on the deflectable portions of the catheter shafts. Multiple tracking electrodes can be employed on the deflectable portions of the catheters for the EAM system 70 to detect and recreate the curvature of the catheters in the body. In one example, each tracking electrode is coupled to a corresponding lead conductor, or lead wire, which extends along the shaft to the proximal portion where it is coupled to an electrical connector. The electrical connector can be coupled to the EAM system 70 such as via cables.

Several constraints are employed in the design and implementation of tracking electrodes and associated lead conductors. Among these constraints include that each tracking electrode and associated lead conductor are to be electrically isolated from one another as well as from other conductive material in the catheter such as a conductive braided member along the length of the shaft. Additionally, precise placement of the tracking electrode on the shaft is desired. For instance, the tracking electrode can be radiopaque, and clinicians can use the tracking electrode to visualize placement of the catheter while using fluoroscopy. Also, electrode location with respect to the catheter elements and interelectrode spacing are programmed parameters in several tracking and mapping software programs, and three-dimensional reconstruction and modeling is performed with electrode spacing as a constraint in the modeling curve. Thus, the design and implementation of catheters using tracking electrodes can benefit from ready access to lumens carrying conductor leads.

The disclosure relates to a catheter having a segment in the braided member that enables a tube carrying a lead conductor to transfer from the main lumen to the outer surface of the braided member. The segment can provide for shaft stiffness, pushability, kink resistance, flexibility, and torque transmission, which can be undesirably affected by removing large portions of the braided member to pass the tube. The catheter includes an elongated shaft defining a lumen along a longitudinal axis and having a distal segment. The elongated shaft includes a longitudinally extending inner member defining the lumen, a support member disposed on the longitudinally extending inner lumen, and a reinforcement member. The support member including a proximal portion and a distal portion separated by a breakout section, and the distal portion includes an outer surface. The reinforcement member is disposed at the breakout section and includes a reinforcement proximal end and a reinforcement distal end. The reinforcement proximal end is coupled to the proximal portion and the reinforcement distal end is coupled to the distal portion. An electrode is disposed on the distal portion of the elongated shaft. An elongated tube extends longitudinally along the elongated shaft. A lead conductor is disposed within the tube and electrically coupled to the electrode. The tube extends longitudinally along the proximal portion radially underneath the proximal portion, extends longitudinally on the outer surface along the distal portion, and extends from underneath the proximal portion to the outer surface at the reinforcement member.

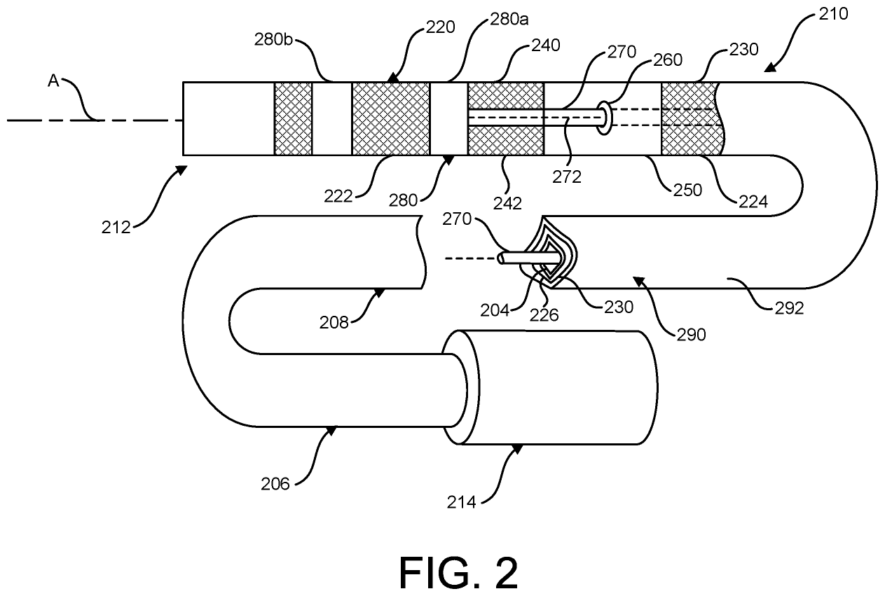

FIG. 2 illustrates an example catheter 200 that can be used in with catheter assembly 100. For instance, catheter 200 can be further configured as a guide catheter, dilator, ablation catheter or other flexible, deflectable medical tool that can be tracked via an impedance tracking system such as EAM system 70. The catheter 200 includes an elongated shaft 202, such as an elongated and flexible shaft 202 defining a longitudinal axis A. The shaft 202 also defines a main lumen 204 along the longitudinal axis A and has a proximal section 206, a longitudinal section 208, and a distal section 210. The distal section 210 includes a distal tip section 212. The proximal section 206 can be coupled to a handle 214 proximal to the shaft 202.

The shaft 202 includes a plurality of components disposed along the distal section 210 and defining a main lumen 204. The shaft 202 includes a support member 224 disposed between a longitudinally extending inner member 226 and a longitudinally extending outer layer 290. The inner member 226 defines the main lumen 204, and the support member 224 is disposed on the inner member 226. In various examples, the inner layer, the outer layer, or both are formed of multiple layers. The support member 224 includes an elongated and flexible proximal portion of the support member, or proximal support member 230 disposed on the inner member 226, and the support member 224 includes a distal portion of the support member, or distal support member 240 disposed on the inner member 226. The distal support member 240 includes an outer surface 242. The distal support member 240 is longitudinally spaced apart from the proximal support member 230 along the axis A at a breakout section of the support member 224. The distal support member 240 is longitudinally spaced-apart from the proximal support member 230 at a suitable distance. In various embodiments, the range of the spaced apart distance can be 1 millimeter to 20 millimeters, for instance, the breakout section is in a range of 2 millimeters to 10 millimeters. In the illustrated example, the proximal and distal support members 230, 240 include a braided fabric, such as a stainless-steel braided fabric. For illustration, the support member 224 and proximal and distal support members 230, 240 are presented as braided member 224 and proximal and distal braided members 230, 240. In some examples, the proximal and distal support members 230, 240 are of different materials. For example, the proximal support member 230 can be of a braided fabric and the distal support member 240 can be of a tubular structure, such as a coiled shaft member or hypotube. Still, in some examples, neither the proximal nor the distal support member 230, 240 is of a braided fabric.

According to various embodiments, a reinforcement member 250 is disposed along the axis A between the proximal support member 230 and the distal support member 240, such as at the breakout section of the support member 224. The reinforcement member 250 is coupled to the proximal support member 230 and the distal support member 240. In some examples, the reinforcement member 250 is of a different material than the proximal support member 230 and the distal support member 240. The reinforcement member 250 can include a plastic or metal tubular member without a braided fabric, a strain relief, a thread wound around the inner member 226 or wound around the support member 224 and the inner member 226, a braid or braided section, or other structure. In some examples, the reinforcement member 250 includes an opening 260, such as a plurality of apertures or openings along a longitudinal side of the reinforcement member 250. For instance, a reinforcement member 250 constructed from a thread wound around the support member 224 and inner member 226 can include an opening between pitches of coil.

An elongated tube 270 extends longitudinally along the shaft 202 and carries a lead conductor 272 that is coupled to an electrode 280 (e.g., a tracking electrode) disposed on the distal portion 208 of the shaft 202. The tube 270 extends longitudinally along the shaft 202 radially underneath the proximal braided member 230 through the proximal braided member 230. In various examples, the proximal braided member 230 includes an inner surface 232. The tube 270 extends longitudinally along the proximal braided member 230 radially underneath the inner surface 232 such as between the inner surface 232 and the inner member 226. The tube 270 extends from underneath the proximal braided member 230 to the outer surface 242 of the distal braided member 240 at the breakout section of the braided member 224. To pass from underneath the proximal braided member 230 to the outer surface 242 of the distal braided member 240, the tube 270 can pass underneath an end of the reinforcement member 250, such as underneath a distal end of the reinforcement member 250, over an end of the reinforcement member 250, such as over a proximal end of the reinforcement member, or through the opening 260 in the reinforcement member 250, and extends longitudinally along an outer surface 242 of the distal braided member 240. The tube 270 can be attached to the outer surface 242 of the distal braided member 240.

According to various examples, the distal portion 210 of the shaft 202 includes a plurality of longitudinally spaced-apart tracking electrodes 280a . . . 280n, and each tracking electrode 280a . . . 280n can be electrically coupled to a respective lead conductor carried in a corresponding tube extending longitudinally along the shaft 202 and terminated at the proximal portion 206. The tracking electrodes 280 are illustrated as ring electrodes in the shaft. In certain examples, the tracking electrodes 280a . . . 280n are configured for use with an impedance-based tracking system. The proximal portion 206 or handle 212 can include an electrical connection that can be coupled to an impedance-based tracking system, such as EAM system 70. In one example, the electrical connection is available under the trade designation LEMO. In some examples, the shaft 202 further includes the outer layer 290 disposed over braided member 224 and tube 270 to form an outermost surface 292 of the shaft 202. The tracking electrodes 280a . . . 280n are exposed on the outermost surface 292 of the catheter 200.

The catheter 200 can include additional components for a selected implementation. In some examples, such as in the case of the catheter 200 configured as a guide sheath or dilator, the proximal portion 206 or handle 214 can include a hemostatic valve that can be coupled to a source of irrigation fluid and a port to receive catheters within the main lumen 204. In some examples, the distal tip section 212 can be configured as a dilator tip. In some examples the shaft 202 can include a liner layer (not shown) coaxial with the main lumen 204 to define an inner wall of the main lumen 204. In examples such as in which the catheter 200 is a guide sheath or dilator, the liner layer can be a thin wall constructed of polytetrafluoroethylene (PTFE). In some examples, such as in the case of the catheter 200 configured as an ablation catheter, the proximal portion 206 or handle can be coupled to a source of ablation energy, such as an electrical signal from console 130 or a cryogenic fluid. For instance, the main lumen 204 can be configured to carry electrical leads, such as leads to the ablation electrodes or other sensors, steering wires, or a conduit of irrigation fluid along the shaft 202 to the distal portion 210. In some examples, the distal tip section 212 can be configured to include an ablation electrode assembly or other sensors.

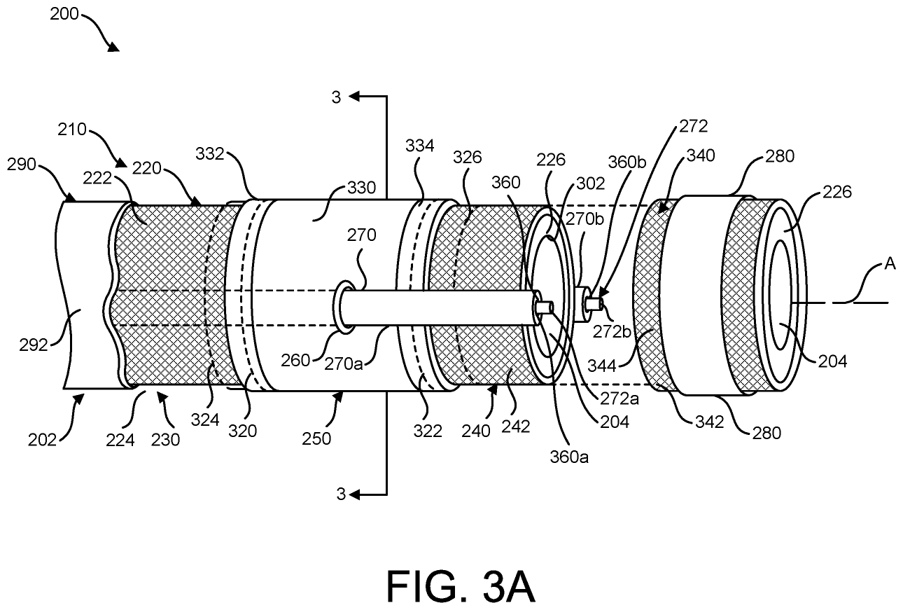

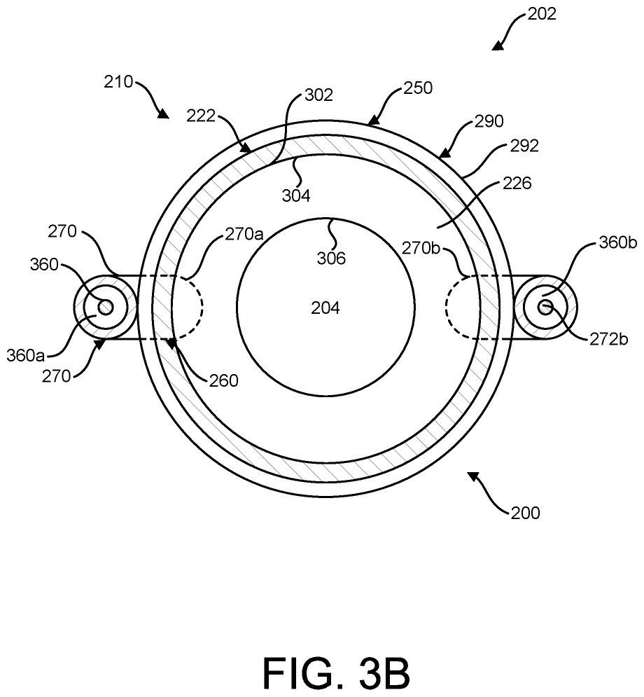

FIGS. 3A and 3B illustrate an example of catheter 200 having a reinforcement member as a tubular structure disposed in the breakout section of the support member between spaced-apart proximal and distal support members. FIG. 3A illustrates features of the catheter 200 from a perspective view, and FIG. 3B includes a cross section front view of the catheter 200, such as the distal portion 210, taken along line 3-3 of FIG. 3A. As shown, the support member is constructed of a braided fabric. In particular, the catheter 200 includes the elongated and flexible shaft 202. The shaft 202 includes the braided member 224 and a longitudinally extending inner member 226, and the outer layer 290, which are coaxial with the main lumen 204. The longitudinally inner member 226 defines the main lumen 204. The braided member 224 can include an inner side 306 disposed toward the main lumen 204 and opposite the outer side 222. The longitudinally extending inner member 226, such as a plurality of longitudinally extending inner layers, are disposed radially underneath the braided member 224. The inner member 226 can include an outer side 304 (radially underneath the inner side 302 of the braided member 224) and an inner wall 306 defining the main lumen 204. In some examples, the shaft 202 can include a plurality of concentric or coaxially braided members along a longitudinal section of the shaft 202, such an innermost braided member and an outermost braided member. In certain examples, a single distal braided member 240 disposed along a longitudinal section of the shaft 202, i.e., a distal braided member 240 that is not concentric with another distal braided member, is also an outermost distal braided member such as the illustrated outermost distal braided member 240.

The braided member 224 of the shaft 202 includes an elongated proximal portion, or proximal braided member 230, disposed on the inner member 226, and the braided member 224 includes a distal portion, or distal braided member 240, disposed on the inner member 226. The distal braided member 240 includes an outer surface 242. The distal braided member 240 is longitudinally spaced apart from the proximal braided member 230 along the axis A as at a breakout section of the braided member 224. In some examples, the spacing between the proximal braided member 230 and the distal braided member 240 is in a range of 2 millimeters to 10 millimeters, such as 5 millimeters. A reinforcement member 250 is disposed along the axis A between the proximal braided member 230 and the distal braided member 240 such as at the breakout section. The proximal braided member 230 is terminated distally at a first braided member distal end 320. The distal braided member 240 is terminated proximally at a second braided member proximal end 322. The first braided member distal end 320 is longitudinally spaced apart from the second braided member proximal end 322. The reinforcement member 250, in various examples, includes a longitudinal surface 330 having an opening 260 and is terminated proximally at a reinforcement proximal end 324 and terminated distally at a reinforcement distal end 326. The reinforcement member 250 is coupled to the proximal braided member 230 and the distal braided member 240. For example, the reinforcement proximal end 324 is coupled to the first braided member distal end 320 and the reinforcement distal end 326 is coupled to the second braided member proximal end 322.

In some examples, the reinforcement member 250 includes an outer surface 330, such as a longitudinal surface 330, and the first braided member distal end 320 is positioned under the outer surface 330 of the reinforcement member 250 such that the reinforcement member 250, and reinforcement proximal end 324, overlaps a distal section of the proximal braided member 230. Also, in various examples, the second braided member proximal end 322 is positioned under the outer surface 330 of the reinforcement member 250 such that the reinforcement member 250, and reinforcement distal end 326, overlaps a proximal section of the distal braided member 240. In other examples, the reinforcement member can be positioned to overlap sections of the proximal, or the reinforcement proximal and distal ends can abut against the first and second braided ends. In some examples, the reinforcement member 250 is attached to the proximal and distal braided members 230, 240, such as by crimping the reinforcement member 250 proximate the reinforcement ends 324, 326. Tubular attachment pieces 332, 334, such as tubular pieces of a polyethylene terephthalate (PET) medical heat shrink tubing, can be attached to the reinforcement member 250 and the proximal and distal braided members 230, 240 at the respective overlapping sections for support. Other examples are contemplated, such as the reinforcement member 250 and the proximal and distal braided members 230, 240 can be welded or adhered together, or the braid ends can be laser cut and welded to reduce the likelihood the braided fibers will unfurl, instead of or in addition to the PET heat shrink tubing.

In examples in which the reinforcement member 250 includes the opening 260 through which the tube 270 extends, the opening 260 is positioned on the reinforcement member 250 so as to be disposed between the spaced apart first braided member distal end 320 of the proximal braided member 230 and the second braided member proximal end 322 of the distal braided member 240. The opening 260 can also be positioned between spaced apart tubular attachment pieces 332, 334.

The proximal and distal braided members 230, 240 can provide characteristics to the catheter 200 such as reduced kinking, wrinkling, or buckling of the shaft 202 and can provide enhanced balance for pushability, deflectability, and torque transmission such as during rotation about the longitudinal axis A. The braided members 230, 240 can be constructed from a woven fabric, such as woven fabric 340 or layer of braided strands or fibers 342 that may form interstitial spaces 344 between the fibers 342. The braided members 230, 240 can further be characterized by the warp and weft and bias of the fibers 342 as well as picks per inch. For instance, in some examples, the picks per inch can remain generally uniform along the entire longitudinal length of one or both of the braided members 230, 240. In some examples, the picks per inch can vary along portions of the longitudinal length of one or both of the braided members 230, 240. The braided members 230, 240 can be constructed from fibers 342 that include stainless steel fibers, such as conductive fibers, or high strength polymer fibers, or as layers of different materials. The braided members 230, 240 can be constructed from the same type or of different types of woven fabric 340.

The reinforcement member 250, in various examples, is a tubular member, such as plastic or metal tube suitable for coupling to the braided members 230, 240 and suitable for attaching to the PET heat shrink pieces 332, 334. In one example, the reinforcement member 250 can be constructed from a plastic extrusion or a laser cut metal tube and fashioned as a frame member or tubular coil member. In some examples, the reinforcement member 250 is constructed to be compatible with a lamination, or reflow, process used to apply the outer layer 290 of the catheter 200. In one example, the reinforcement member 250 includes a plurality of holes or is an open structure, in addition to the opening 260, through which the plastic of the outer layer 290 can flow during the lamination process to provide for added strength. The size of the reinforcement member 250, such as the outside diameter, can be selected to not appreciably increase the outside diameter of the shaft 202, and the length of the reinforcement member 250 can be selected to allow for sufficient structural support between the longitudinally spaced apart proximal and distal braided members 230, 240 without appreciably affecting desirable characteristics of the shaft 202.

The elongated tube 270 extends longitudinally underneath the proximal braided member 230. In one example, the elongate tube 270 interfaces with the inner surface 232 of the proximal braided member 230 as the tube 270 traverses the proximal braided member 230. The elongated tube 270 interfaces with an outer surface 242 of the distal braided member 240. For example, the elongated tube 270 is abutted against the outer surface 242 of the distal braided member 240 or is glued (e.g., using an adhesive) or otherwise attached to the outer surface 242 of the distal braided member 240. For instance, a longitudinally extending coiled thread, such as a nylon thread or twine, can be wrapped around the braided member 224, reinforcement member 250, and tube 270 to hold the tube 270 against a least the distal braided member 240. In one example, the tube 270 can extend from within the reinforcement member at a distal end 326 of the reinforcement member 250 to the outer surface 242 of the distal braided member 240 in the case of the reinforcement member 250 overlapping the proximal and distal braided members 230 240. In another example, the tube 270 can travel through the opening 260 from underneath the proximal braided member 230 to the outer surface 242 of the distal braided member 240. In one example, the elongated tube 270 extends longitudinally and generally straight along the proximal braided member 230 and the distal braided member 240. The elongated tube 270 is configured from a suitable material, such as a polymer, and defines a longitudinally extending lead lumen 360 along the length of the tube 270. The tube 270 is configured to carry a lead conductor, such as lead wire 272, within the lead lumen 360. The lead wire 272 is configured to be electrically coupled to a tracking electrode 280 and carry an appropriate electrical signal from the electrical connector at the proximal portion 204 to the tracking electrode 280. In one example, the elongate tube 270 is of a substantially smaller diameter than the shaft 202, and the main lumen 204 has a substantially larger diameter than the diameter of the lead lumen 360.

In an example of a catheter 200 including multiple tracking electrodes, an elongated tube carrying a lead wire is included for each tracking electrode. Each of the tubes extend longitudinally along the proximal braided member 230 radially underneath the proximal casing member 230 and longitudinally on the outer surface 242 along the distal braided member 240, and extends to the outer surface 242 at the reinforcement member 250. In one example, the reinforcement member 250 can include an opening for each elongate tube 270. In some examples, the catheter 200 can include two tracking electrodes and includes a first elongated tube 270a carrying a first lead wire 272a within first lead lumen 360a and a second elongated tube 270b carrying a second lead wire 272b within first lead lumen 360b. Multiple elongated tubes 270 can be radially spaced-apart radially underneath the proximal braided member 230 and on the outer side 242 of the distal braided member 240. The reinforcement member 250 can include radially spaced-apart openings. For example, the elongated tubes 270a, 270b and apertures are radially spaced 180 degrees apart. The lead wires 272 can be disposed within the elongated tubes 270 either as bare wire or with an insulation covering. Each elongate tube 270 can terminate proximate the corresponding tracking electrode.

The outer layer 290 can be disposed on the proximal braided member 230, the reinforcement member 250, and the distal braided member 240 and also on the tube 270 as disposed on the outer surface 242 of the distal braided member 240. In some examples, the outer layer 290 can be formed as a coating of reflowable plastic or thermoplastic material that extends over the braided member 224 and seals underlying components of the shaft 202. For instance, the coating can seep by reflowing over braided material of the proximal and distal braided members 230, 240 such as over the fibers 342 and into the interstitial spaces 344 of the braided material 340, onto the tubes 270, and onto the reinforcement member 250. A thicker wall of the outer layer 290 over the breakout section or between the proximal and distal braided members 230, 240, helps to strengthen the shaft 202 and resist the appearance of stress lines. In one example, the outer layer 290 is a polyether block amide and, in some examples, is available under the trade designations PEBAX from Arkema, S.A., and VESTAMID E from Evonik Industries, AG.

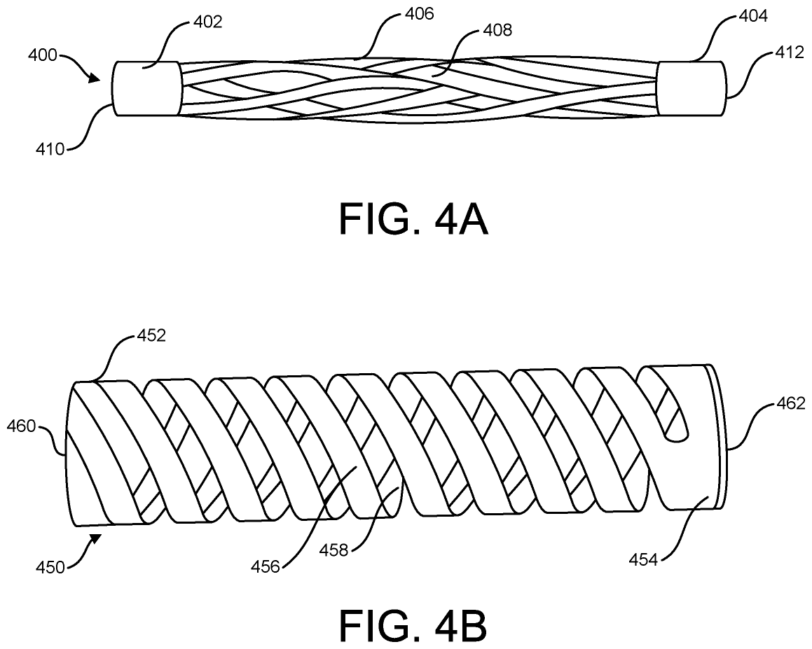

FIG. 4A illustrates an example framed reinforcement member 400 as an example of the reinforcement member 250. The framed reinforcement member 400 includes proximal annular member 402, a distal annular member 404, and a plurality of cross members 406 extending between the annular members 402, 404 and forming openings 408 between the cross members 406. The annular members 402, 404 are configured to be disposed on the axis A and surround the braided members 230, 240. The cross members 406 longitudinally space the annular members 402, 404 along the axis A. The proximal annular member 402 includes reinforcement proximal end 410 and suitable for coupling to the proximal braided member 230. The distal annular member 404 includes reinforcement distal end 412 and suitable for coupling to the distal braided member 240. For example, the first braided member distal end 320 can fit under the proximal annular member 402, and the proximal braided member 230 can be attached to the framed reinforcement member 400 with a PET heat shrink. The second braided member proximal end 322 can fit under the distal annular member 404, and the distal braided member 240 can be attached to the framed reinforcement member 400 with a PET heat shrink. Another example can include the proximal and distal braided members over the ends of the tubular reinforcement member 400. With the proximal and distal braided members 230, 240 under the annular members 402, 404, the tube 270 can extend from the reinforcement member 400 from under the distal annular member 404 over the outer surface of the distal braided member 240. The annular members 402, 404 can be crimped on the braided members 230, 240 or braided members 230, 240 and tube 270. An opening of openings 408 between cross members 406 can be used as opening 260 to extend the tube 270 through the reinforcement member 400. Multiple tubes can be extended through an opening 408 between cross members 406, or each tube of multiple tubes can correspond with an opening of the multiple openings 408. The spacing of the cross members 406 can be selected such that tube 270 can fit through the openings 408 without additional modification or drilling to form a larger gap. The framed reinforcement member can be constructed of a plastic or metal, such as stainless steel, as distinguishable from the braided material, and be either rigid or flexible. The number of cross members 406 can be selected based on a desired rigidity, flexibility, size of aperture, or other characteristics. In examples, the framed reinforcement member 400 can be formed from a laser machined metal tube or an extruded plastic tube.

FIG. 4B illustrates an example tubular double-coiled reinforcement member 450 as an example of the reinforcement member 250. The doubled-coiled reinforcement member 450 includes proximal annular member 452, a distal annular member 454, and a plurality of coil members, such as coil members 456a, 456b extending between the annular members 452, 454 and forming openings 458 between the coil members 456a, 456b. The annular members 452, 454 are configured to be disposed on the axis A and surround braided members 230, 240. The coil members 456 are coiled around axis A at a selected pitch to longitudinally space the annular members 452, 454 along the axis A. The proximal annular member 452 includes reinforcement proximal end 460 and suitable for coupling to the proximal braided member 230. The distal annular member 454 includes reinforcement distal end 462 and suitable for coupling to the distal braided member 240. For example, the first braided member distal end 320 can fit under the proximal annular member 452, and the proximal braided member 230 can be attached to the coiled reinforcement member 400 with a PET heat shrink. The second braided member proximal end 322 can fit under the distal annular member 454, and the distal braided member 240 can be attached to the coiled reinforcement member 450 with a PET heat shrink. Another example can include the proximal and distal braided members over the ends of the tubular reinforcement member 450. With the proximal and distal braided members 230, 240 under the annular members 452, 454, the tube 270 can extend from the reinforcement member 450 from under the distal annular member 454 over the outer surface of the distal braided member 240. The annular members 452, 454 can be crimped on the braided members 230, 240 or braided members 230, 240 and tube 270. The openings 458 between coil members 456 can be used as aperture 260 to extend the tube 270 through the reinforcement member 450. Multiple tubes can be extended through the opening 408 between cross members 456 such as radially or longitudinally spaced apart on opening 458. The spacing of the coil members 456 can be selected such that tube 270 can fit through the openings 458 without additional modification or drilling to form a larger gap. The framed reinforcement member can be constructed of a plastic or metal, such as stainless steel, as distinguishable from the braided material, and be either rigid or flexible. The width, spacing, pitch and number of cross members 406 can be selected based on a desired rigidity, flexibility, size of aperture, or other characteristics. In examples, the coiled reinforcement member 450 can be formed from a laser machined metal tube or an extruded plastic tube.

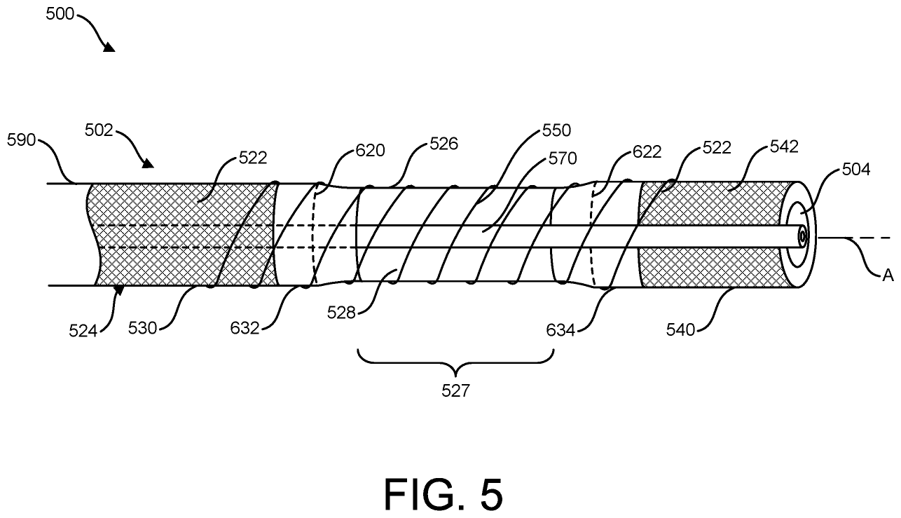

FIG. 5 illustrates an example distal section of catheter 500 having a coiled or wound thread as reinforcement member 550 disposed between spaced-apart proximal and distal support members. The support members are illustrated as braided members formed from a braided material. A shaft 502 includes a braided member 524 disposed radially over a longitudinally extending inner member 526, and an outer layer 590, which are coaxial with a main lumen 504. The longitudinally inner member 526 defines the main lumen 504. The braided member 524 can include an inner side disposed toward the main lumen 504 and opposite an outer side 522. The longitudinally extending inner member 526 is disposed radially underneath the braided member 524. The inner member 526 can include an outer side 528 (radially underneath the inner side of the braided member 524) and an inner wall defining the main lumen 504.

The braided member 524 includes an elongated proximal braided member 530 disposed on the inner member 526, and the braided member 524 includes a distal braided member 540 disposed on the inner member 526. The distal braided member 540 includes an outer surface 542. The distal braided member 540 is longitudinally spaced apart from the proximal braided member 530 along the axis A on a breakout section 527 of the braided member 524. The proximal braided member 530 is terminated distally at a first braided member distal end 620. The distal braided member 540 is terminated proximally at a second braided member proximal end 622. The first braided member distal end 620 is longitudinally spaced apart from the second braided member proximal end 622. The distance between the proximal and distal braided members 530, 540, such as the distance between the first braided member distal end 620 and the second braided member proximal end 622, can be a suitable distance, such as between 2 millimeters to 10 millimeters. Tubular attachment pieces 632, 634, such as tubular pieces of PET heat shrink tubing, can be attached to the proximal and distal braided members 530, 540 such as over the first braided member distal end 620 and the second braided member proximal end 622 to further secure the braided member 524 to the inner member 526.

The tube 570 extends from radially underneath the proximal braided member 530 and tubular attachment piece 632, such as between the inner side of the proximal braided member 530 and the outer side 528 of the inner member 526 at the breakout section 527 to be radially over outer side 542 of the distal braided member 540 (and can be over tubular piece 634 as illustrated). The reinforcement member 550 is constructed from a thread, such as a filament, twine, or braid, wound around or coiled around the proximal and distal braided members 530, 540, inner member 526, and tube 570 as illustrated, in which the thread is wound at a selected pitch and tension. For instance, the reinforcement member 550 can include a nylon-12 thread having suitable strength and melting temperature to sustain further manufacturing processes. The reinforcement member 570 can be wound over the proximal braided member 530, over the tube 570 extending from the distal end 620 of the proximal braided member 530, over the tube 570 and inner member 526, and over the tube 570 and the outer side 522 of the distal braided member 540. The thread can be terminated on the outer side 522 of the braided member on both the proximal and distal braided members, and the tube 570 can further extend distally past the thread. In one example, the pitch or tension of the thread can be different over the proximal and distal braided members 530, 540 than over the breakout section 527. For instance, the pitch can be increased over the breakout section 527 than over the proximal and distal braided members 530, 540. In one example, multiple threads can be used to form the reinforcement member 550, or structures can be disposed in the breakout section 527, such as metal slats extending across the breakout section 527 and covered by a thread to form the reinforcement member 550. In one example, the proximal and distal support members 530, 540 are sufficiently close together but still spaced-apart enough to permit the tube 570 to extend through the breakout section 527 without a thread wound around the braided members or breakout section.

Features of the catheter similar to features catheter 200 can be constructed similarly. For example, the braided member 524 can be configured as described above. The elongated tube 570 is configured from a suitable material, such as a polymer, and defines a longitudinally extending lead lumen along the length of the tube 570. The tube 570 is configured to carry a lead conductor, such as lead wire, within a lead lumen. The lead wire is configured to be electrically coupled to a tracking electrode and carry an appropriate electrical signal from the electrical connector at the proximal portion of the catheter to the tracking electrode.

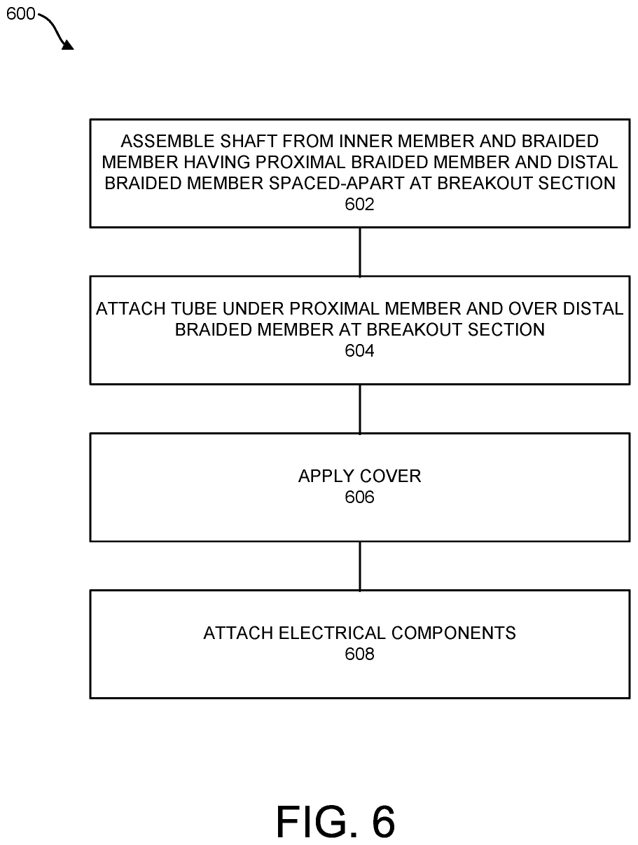

FIG. 6 illustrates an example method 600 of manufacturing a catheter. In one example, method 600 includes a method of constructing a catheter shaft defining a lumen and attached to a tube defining a lead lumen including a conductor lead configured to be attached to a tracking electrode exposed on the catheter shaft. In one example, the elongate braided member is formed over an inner member, the braided member having a proximal braided member longitudinally spaced apart from a distal braided member by a breakout section at 602. In one example, an elongated braided member, such as braided member 224 is formed by coupling a proximal braided member and a distal braided member to reinforcement member having an opening. The proximal and distal braided members can include an electrically conductive stainless steel braided material. The reinforcement member is a tubular member that, in one example, does not include a braided material. An elongated tube extending longitudinally is attached along the elongated braided member, the tube disposed longitudinally along the proximal braided member and radially underneath the proximal braided member, to the breakout section, and then extending along the outer surface of the distal braided member at 604. In an example of a reinforcement member, the tube is disposed longitudinally along the proximal braided member and radially underneath the proximal braided member, extending through the opening in the longitudinal surface of the reinforcement member, and then extending along the outer surface of the distal braided member. In another example of a reinforcement member the tube is disposed longitudinally along the proximal braided member and radially underneath the proximal braided member, extending through the breakout section, and then extending along the outer surface of the distal braided member. A thread is wound on the braided member and the inner member and tube at the breakout section. The tube and braided member receive a cover (or outer layer) at 606. In one example, a thread, such as a nylon thread, can be longitudinally coiled around the tube and braided member to hold the tube against the distal braided member. A distal end of the tube is sealed to protect the lead lumen while the outer layer is being applied. The distal end of the tube is sealed to reduce the likelihood of the outer layer filling the lead lumen. Additionally, the distal end of the tube can be reinforced to remain under tension against the distal braided member in preparation for the outer layer. In one example, the distal end of the tube is secured to outer surface of the distal braided member via an attachment member including tape, such as silicone tape, melted extrusions, or a heat shrink tubing applied over the ends and heated. An example of the heat shrink tubing can include PET medical heat shrink tubing. The cover (or outer layer) can be applied via reflow. The attachment member can be removed after the outer layer is applied. A lead conductor is put into a lead lumen of the tube, and the lead conductor is electrically coupled to a tracking electrode disposed on the shaft at 608.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter, comprising:
an elongated shaft defining a lumen along a longitudinal axis and having a distal portion; the shaft comprising:
a longitudinally extending inner member defining the lumen;
a support member disposed on the longitudinally extending inner member, the support member including a proximal portion and a distal portion separated by a breakout section, the distal portion having an outer surface; and
a reinforcement member disposed on the breakout section and having a reinforcement proximal end and a reinforcement distal end, the reinforcement proximal end coupled to the proximal portion and the reinforcement distal end coupled to the distal portion;
an electrode disposed on the distal portion of the elongated shaft;
an elongated tube extending longitudinally along the elongated shaft; and
a lead conductor disposed within the tube and electrically coupled to the electrode;
wherein the tube extends longitudinally along the proximal portion radially underneath the proximal portion, extends longitudinally on the outer surface along the distal portion, and extends from underneath the proximal portion to the outer surface at the reinforcement member.

2. The catheter of claim 1, wherein the support member is formed of a braided material.

3. The catheter of claim 1, wherein the proximal braided member and the distal braided member include conductive fibers.

4. The catheter of claim 1, wherein the elongated shaft further comprises a reinforcement member having a tubular longitudinal side, a reinforcement proximal end, and a reinforcement distal end, the reinforcement proximal end operably coupled to the proximal portion and the reinforcement distal end operably coupled to the distal portion.

5. The catheter of claim 4, wherein the reinforcement member includes one of a framed member having a plurality of first annular members attached to and spaced apart by a plurality of longitudinally extending cross members and a tubular coiled member having a plurality of second annular members attached to and spaced apart by a longitudinally extending coil.

6. The catheter of claim 5, wherein the reinforcement member is formed from a laser machined tube.

7. The catheter of claim 4, wherein the longitudinal side extends along the axis and an opening is formed on the longitudinal side, and wherein the tube extends from underneath the proximal portion to the outer surface through the opening of the reinforcement member.

8. The catheter of claim 1, wherein the reinforcement member includes a thread wound around the tube and breakout section.

9. The catheter of claim 1, wherein the shaft includes an outer layer disposed on the support member and the reinforcement member.

10. The catheter of claim 1, wherein the elongate tube includes a plurality of elongate tubes radially spaced-apart on the outer side of the distal portion.

11. The catheter of claim 1, wherein the inner member includes an inner surface, and the inner surface includes a liner layer.

12. The catheter of claim 1, wherein the catheter includes one of a dilator and a guide catheter.

13. The catheter of claim 1, wherein the shaft includes a distal tip section having an ablation electrode assembly.

14. The catheter of claim 1, wherein the electrode is a ring electrode.

15. A method of manufacturing a catheter, the method comprising:
forming a distal portion of an elongated member from an inner member defining a lumen, a proximal braided member, and a distal braided member spaced apart from the proximal braided member at a breakout section, the proximal braided member and distal braided member disposed on the inner member;
attaching an elongated tube extending longitudinally along the elongated member and within an inner side of the proximal braided member, extending longitudinally along an outer side of the distal braided member, and through breakout section;
forming a reinforcement member on the breakout section; and
disposing an outer layer on the elongated member to form a shaft.

16. The method of claim 15, wherein the reinforcement member is formed by winding a thread over the elongated member.

17. A system for tracking a catheter during an electrophysiology procedure on a patient, the system comprising:
a patch electrode mechanically couplable to the patient;
a catheter disposable within a patient, the catheter comprising:
an elongated shaft defining a lumen along a longitudinal axis and having a distal portion; the shaft comprising:
a proximal braided member comprising a first braided member distal end;
a distal braided member having an outer surface and a second braided member proximal end, the second braided member proximal end longitudinally spaced-apart from the first braided member distal end; and
a reinforcement member having an aperture, a reinforcement proximal end, and a reinforcement distal end, the reinforcement proximal end operably coupled to the first braided member distal end and the reinforcement distal end operably coupled to the second braided member proximal end;
an electrode disposed on the distal portion of the elongated shaft;
an elongated tube extending longitudinally along the elongated shaft; and
a lead conductor disposed within the tube and electrically coupled to the electrode;
wherein the tube extends longitudinally along the proximal braided member radially underneath the proximal braided member, extends longitudinally on the outer surface along the distal braided member, and extends from within the lumen to the outer surface through the aperture of the reinforcement member; and
a controller operably coupled to the patch electrode and the tracking electrode, the controller configured to receive the electrical signal and determine a position of the catheter with respect to the patient.

18. The system of claim 17, wherein the controller if further configured to generate an electroanatomical map of the heart of the patient.

19. The system of claim 17, wherein the controller is configured to determine the position of the catheter via an impedance-based process.

20. The system of claim 17, wherein the reinforcement member is of a material dissimilar from the proximal braided member and the distal braided member.
